(19) United States
(12) Patent Application Publication
(10) Pub. No.: US 2024/0328936 A1
(43) Pub. Date: Oct. 3, 2024

(54) APPARATUS, SYSTEMS AND METHODS FOR USE IN OPTICAL GAS ABSORPTION MEASUREMENTS

(71) Applicant: Servomex Group Limited, Crowborough (GB)

(72) Inventors: Michael Lawson, Crowborough (GB); Bahram Alizadeh, Crowborough (GB); Martin Lopez, Crowborough (GB); Michael Ratcliffe, Crowborough (GB)

(73) Assignee: Servomex Group Limited, Crowborough (GB)

(21) Appl. No.: 18/619,884

(22) Filed: Mar. 28, 2024

(30) Foreign Application Priority Data
Mar. 31, 2023 (GB) 2304895.2
Dec. 29, 2023 (GB) 2320141.1
Mar. 27, 2024 (GB) 2404425.7

(51) Int. Cl. G01N 21/3504; G01J 3/433; G01N 21/39

(52) U.S. Cl. CPC G01N 21/3504; G01J 3/433; G01N 21/39; G01N 2021/399

(57) ABSTRACT

An apparatus for optical gas detection and/or measurement in an absorption spectroscopy system comprising: a gas cell for containing a gas sample or calibration gas with at least one gas exchange port and at least one optical element for allowing transmission of electromagnetic radiation of a desired wavelength range in and out of the gas cell; and at least one source of converging, diverging or collimated electromagnetic radiation for transmitting electromagnetic radiation through a gas sample contained within the gas cell and towards at least one detector; said detector to monitor absorption of electromagnetic radiation for at least one absorption wavelength or wavelength range associated with at least one gas species, by detecting transmitted electromagnetic radiation that is not absorbed; at least one analyser for analysing an output signal from the at least one detector to measure a parameter of at least one gas species within the gas sample, characterised in that at least one dead volume within the spectrometer is filled with a transmissive filler material that reduces or eliminates the presence of at least one undesired gas species.

### CROSS REFERENCE TO RELATED APPLICATIONS

This patent application is entitled to claim priority from UK Patent Application No. 2404425.7 filed on Mar. 27, 2024, UK Patent Application No. 2320141.1 filed on Dec. 29, 2023, and UK Patent Application No. 2304895.2 filed on Mar. 31, 2023, which are incorporated herein by reference.

### FIELD OF THE INVENTION

This invention relates to apparatus and materials for use in gas absorption spectroscopy in general, and in tuneable diode laser absorption spectroscopy (TDLS) and non-dispersive infrared spectroscopy in particular. The invention has applications in the detection and measurement of one or more species in a gas, such as those produced by an artificial or natural process such as an industrial, medical or physiological process.

### BACKGROUND OF THE INVENTION

An example absorption measurement system consists of a source of electromagnetic radiation such as a tuneable laser source, for example a tuneable diode laser (TDL), or a broadband source such as an incandescent source or light emitting diode (LED) in combination with a wavelength range selective element, such as an optical passband filter or grating. The source emits a beam of electromagnetic radiation that is focussed on to a detector, which may be a solid state photovoltaic, photoconductive, photomultiplier, pyrometer, thermopile or bolometer detector. The substance that is to be analysed is positioned between the electromagnetic radiation source and the detector, so that the electromagnetic radiation incident on the detector may be modified (i.e. reduced by absorption) by its path through the substance. The modifications to the electromagnetic radiation enable various parameters of the measurand gas or other substance to be determined by a signal processing system that is coupled to the detector. Such measurements may take place across an in-situ gas sample measurement volume, such as a process stack, or within a measurement gas sample cell. FIG. 1 illustrates a basic extractive absorption measurement apparatus, where 101 is the source of electromagnetic radiation, 102 is the sample cell with transmissive windows at each end and gas inlet 103 and outlet 104, and the detector 105. A signal processing system 106 is used to process the detector signal and produce the measurand concentration based on a calibrated algorithm. In some cases, the substance to be analysed is a gas produced by an industrial, medical or natural process and the measurand may be a parameter of one or more chemical species that are present in this gas. References to a ‘measurand gas’ or ‘measurand species’ in this patent specification are intended to refer to a gas or gas species for which one or more parameters are to be measured or detected; the ‘measurand’ is the presence of a gas species or a measurable parameter of the gas species. Examples of measurand species include, but are not limited to, gaseous water, O2, NO, NO2, CO, CO2 and hydrocarbons such as methane or ethane. These measurements are often used for optimising process efficiency and for the monitoring and/or minimising of the production of pollutants and greenhouse gases, as well for the monitoring and/or optimisation of physiological well-being. The presence and/or amount fraction (concentration) of one or more of these measurand species may be determined by absorption spectroscopy measurements using one or more TDLs. It is known to convert observed changes in electromagnetic radiation intensities to useful physical parameters, such as concentrations and temperatures, but this typically requires a series of assumptions to be made regarding the measurand species and measurement apparatus. The term electromagnetic radiation covers a very broad wavelength range and often, but not exclusively, absorption spectroscopy measurements occur in the ultraviolet, visible and infrared regions of the electromagnetic spectrum. Illustrations within this patent will be given for the infrared light region of the electromagnetic spectrum, for obtaining absorption spectroscopy measurements corresponding to certain molecular vibrational energy transitions, but the same principles may be applied to other relevant wavelength regions and should not be considered limited to this spectral region. For case of expression, throughout this patent specification, the terms electromagnetic radiation or light may be used interchangeably and should be interpreted as being equally applicable to the ultraviolet, visible, infrared or other relevant range of the electromagnetic spectrum unless stated otherwise.

In operation of an example laser gas analyser system, the wavelength of the beam emitted by a TDL is scanned over a range of wavelengths including one or more absorption lines of the measurand gas species. At specific wavelengths within the range of wavelengths scanned, light is absorbed by the measurand gas, and these spectral absorption lines can be detected by measuring changes in the light flux through the substance to be analysed.

In operation of an example broadband gas analyser system, the source emits over a relatively broad wavelength range (encompassing at least one relevant absorption line), even if limited via the use of an optical passband filter or diffraction grating, and the throughput intensity is modified by absorption simultaneously occurring over one or more absorption lines of the measurand gas species. The integrated effect of the spectral absorption line(s) can be detected by measuring changes in the light flux through the substance to be analysed due to the presence of the measurand.

Absorption lines have characteristic “shapes” in wavelength space that are dependent on the intrinsic physical properties of the gas species (bond angles, bond lengths, number of electrons), as well as the extrinsic physical properties (velocity, temperature) and properties of the environment (pressure, composition of surroundings, etc.). The following paragraphs attempt to give a brief overview of the mechanisms by which these shapes are produced and to give some insight about current practical limitations concerning the recovery of useful properties, such as concentration and temperature, where other perturbing factors are not, or cannot be, well-defined.

In the context above, an absorption “line” is an observable change in light transmission, which coincides with a frequency (wavelength) interval over which a photon may induce a gas molecule to transit from one quantum state to another. The probability that a photon with a particular wavelength and polarisation will elicit transition of quantum states is given by the transition's absorption cross section. The transition dependent cross section can be estimated from first principles, but, in practice, it is typically measured experimentally with high accuracy.

Since the interaction of light with matter is inherently quantum in nature, the degree to which a measurand gas species with a fixed chemical identity absorbs light of a given frequency is determined not only by its number density in the measurement path but also by its precise quantum mechanical state. For any given gas species, there will exist numerous quantum states with spectroscopically distinct absorption properties.

From the Heisenberg uncertainty principle, it follows that the energies of quantum states cannot be precisely defined. This uncertainty causes a smearing of the photon energies required to elicit a transition between two states, preventing spectral lines from becoming infinitely narrow. The statistical impact of different broadening mechanisms can be gathered into a frequency dependent term referred to as the “spectral line-shape”.

As mentioned earlier, the energies of a molecule's quantum states can be perturbed by its physical environment. For example, outside of a perfect vacuum, molecules collide with other particles. These collisions, like photons, can induce changes in quantum state of the molecule, such that the natural lifetime of the original states is shortened. In gaseous states, the influence of this collisional broadening, to a great extent, resembles natural broadening and, accordingly, its influence is generally treated as a modification to the Lorentzian full width half maximum (FWHM) height. However, in contrast to natural line broadening, the pressure contribution has a more complex relationship with the absorption line shape. Pressure broadening, which is applicable to gases only, for a given set of conditions, can be deduced by physically measuring the line shape and carefully subtracting other known broadening mechanisms. This is often impractical and frequently the collisional broadening is only catalogued at “Normal Temperature and Pressure” (NTP) in the absence of other gases and in air. The resultant values may then be fed into mathematical models, so that linewidths corresponding to different conditions may be extrapolated.

The thermal motion of a measurand gas will cause its constituent molecules to possess a range of velocities in relation to the light source. If one considers these molecules to be “observers” of incoming photons, these photons will appear red or blue shifted. At any given temperature, to conserve energy and momentum, lighter molecules will travel faster, on average, compared to those which are more massive. Therefore, thermal broadening has a positive relationship to temperature and an inverse relationship to molecular mass.

From the paragraphs above, it follows that for most practical spectroscopic applications, where temperature and pressure have an appreciable effect, the resultant transition profile is neither completely Lorentzian nor Gaussian in shape. For this reason, it is known to make use of a “Voigt” profile, which is a convolution of the Lorentzian and Gaussian line shapes.

It is possible to accurately determine, for example, changes of the volume density of a measurand gas in a straightforward fashion, provided the perturbing factors listed above are stable and there are no variations in incident light intensity and measurement path length. Such variations may occur, for example, during thermal flexing and mechanical vibrations of the measurement chamber. Variations in incident light intensity may be caused by a number of factors other than absorbing molecular density changes. For example, variations can be caused by intrinsic fluctuations in the laser output, changes in ambient light intensity levels and/or obscuration in the process sample stream, which may be caused by any combination of dust, tar, corrosion or optical beam misalignment. In an extractive or laboratory setting such environmental factors may be limited, for example, by chemical and/or physical filtration to eliminate dust and/or other physical contamination and the presence of undesired chemical species such as water, or mitigated, for example, by compensating for or controlling the ambient temperature and/or pressure. Even in this situation, compensation in light intensity fluctuations may still be desirable due to source instability or drift (as described, for example, in U.S. Pat. No. 9,546,902). The inventors of the present invention have provided a solution that is particularly well-suited for an in-line, extractive environment such as in a laboratory or equivalent environment, although it could also be employed in an in-situ measurement, such as a pipe or stack.

A further cause of measurement uncertainty can be present due to spatial averaging. This type of uncertainty arises due to the fact that the spectroscopic measurements described above reduce the state of a 3-dimensional system to a 1-dimensional transmittance value. This means that any variations over the pathlength will be averaged, so that all spatial information is completely lost. Assuming that these parameters are not constant along the measurement pathlength, which is especially the case for in-situ gas measurements, this can present major challenges for useful signal recovery.

Direct and indirect cross interference may also occur. This interference results from the effect that the partial pressure of a foreign gas has on the measurement, either through “direct” overlap of absorption lines within the measured wavelength range or from the “indirect” effect of the foreign gas colliding with the measurand gas and modifying the measurand gas absorption line. The composition of the measurement stream is seldom known with precision; if it were, the requirement for the measurement would be nullified and therefore it follows that the line shape produced by a fixed quantity of measurand gas may vary unpredictably if the partial pressures of foreign, broadening gases are not known. Direct cross interference, when caused by a foreign gas, will always result in some degree of additional indirect cross-interference. Depending on the spectroscopic technique used, this may have a significant impact on the measured transition intensity. For example, where wavelength modulation spectroscopy (WMS) is employed, the recovered line intensity is dependent on the ratio of applied amplitude modulation and transition linewidth. It follows that indirect cross interference, in this situation, necessitates a measurement technique whereby the line shape is constantly measured and used to normalise fluctuations in the transition intensity. Any uncertainty in the line shape arising from, for example, electronic noise, will be coupled into the measurement signal, to the detriment of its precision and accuracy. Alternatively, a measurement from a secondary sensor may be used as a normative input although, once again, uncertainties still couple into the measurement and multiple sensors may be needed to achieve full coverage of interferents.

Another potential cause of fluctuations in the optical detector signal, which is not due to direct fluctuations in the ambient light or laser output signal, is the occurrence of constructive and destructive optical interference (etalons) causing an oscillation in the detector signal as the laser is scanned across the measurement wavelength range. Optical interference will even be present, to some degree, with an incoherent broadband light source, due to random effects, but the use of coherent laser light, for example, means that any reflections at any optical surfaces or interfaces along the optical path from the laser output to the detector surface (for example from surfaces/interfaces such as, but not limited to, windows, lenses and reflective interfaces), lead to the production of reflected light with a phase difference in comparison with the incident light, hence leading to optical interference, where the light rays interact. The phase relationship between this reflected light and the incident light may change with time due to such factors as temperature, vibration and pressure fluctuations, since these factors may cause changes in physical dimensions, density or refractive index.

The detector is integrating this optical interference when producing an intensity signal. Since the phase difference will vary with wavelength along the measure path, the symptoms of this optical interference (or etalons) are typically the production of oscillations on the signal baseline as the laser output is scanned across the wavelength measurement range. These combine with other distortions and cause measurement inaccuracies. The signal “baseline” is the signal that would be seen even if no absorbing signal were present, in other words, the “zero absorption” signal. This baseline signal is superimposed on the actual absorption signal when present. In an ideal world, the baseline would be a straight line (flat line centred at zero in perfect circumstances), but in practice this is never achieved. The baseline may not be perfectly flat across the scan range and may have fluctuations and other distortions (or “noise”), which may be of a random or systematic nature and include the above-mentioned oscillations. These oscillations may also be referred to as “fringe” signals in the case of optical interference. These various distortion effects, of whatever origin, lead to increased uncertainty in the determination of the absorption signal or signals, and hence increased uncertainty in the derived molecular density or concentration of the measurand gas. The uncertainty is compounded where the presence of indirect cross-interference necessitates the measurement of line shape in addition to transition intensity, since the periodic etalon fringes may cause the recovered signal to broaden or narrow.

Methods to decrease such optical interference include reducing reflective or partially reflective surfaces in the light path from source to detector that may form etalons, such as by minimising the number of optical components, using wedge windows rather than parallel face windows or using anti-reflection coating optimised for the desired wavelength range. However, it is impossible, in practice, to eliminate this interference effect by reducing reflective surfaces. Where a multi-pass cell is used, interference is unavoidable, as the beam path within a multi-pass cell will always create some amount of optical interference, which is usually significant when the cell is being used for trace level measurements.

Another method to reduce the impact of optical interference on the baseline is to measure and record a reference baseline when no measurand gas is present. This reference baseline may then be subtracted from the live signal to produce a cleaner signal to process. Whilst this may give an immediate improvement to the measurand determination uncertainty, it does not address oscillations on the baseline under changes in ambient conditions (particularly temperature) and hence the effectiveness of this technique is limited.

Another method involves the use of a piezo electric element or similar means to oscillate an active optical element such as a lens or mirror in the optical path. This has the effect of continuously varying the optical pathlengths and hence the phase variations and resultant optical interference. This results in blurring or smoothing down the periodic oscillation on the baseline, through integration over time of the interference fringes formed and therefore reducing the overall effect. However, it adds complexity and cost and suffers from several problems due to using a moving element, such as reduced component lifetime and mechanical failure and, in practice, does not eliminate the problem completely. Moreover, most piezo electric elements require a sufficiently high voltage supply that makes operation in flammable hazardous areas unsuitable.

Other methods make use of the distinct transition line shapes, described earlier, and their difference from the periodic intensity fluctuations arising from optical self-interference. This might be carried out, for example, by least squares fitting a recovered spectrum to a suitable basis-vector which would resemble the expected spectrum in the absence of interference. This method has the advantage that further basis vectors can be employed that resemble the individual spectra of other cross-interfering gases in the measurement. Alternatively, a similar procedure may be carried in the frequency domain using convolution with a suitable kernel function, such as described in U.S. Pat. No. 10,234,378. These methods, however, are limited in that they require assumptions to be made regarding the transition line shape. If it deviates from the expected value, the resulting measurements become inaccurate. For example, if a direct-cross interferent is present in the measurement stream which is not accounted for in the corrective basis-set or frequency domain kernel, such approaches can be detrimental to measurement accuracy. Furthermore, if factors affecting the production of optical noise differ significantly from expected values, such as the free spectral range of the resultant etalon fringes, the efficacy of such approaches is reduced.

A recent novel method (described in UK Patent Application No. GB2113699.9) involves the application of electrical and/or magnetic fields to modify the absorption line characteristics due to the Zeeman and/or Stark effects, whilst leaving the optical interference effects unchanged. In this way, the baseline effect of the optical interference may be deduced and/or decreased or eliminated.

Under constant ambient conditions of pressure, temperature and background gas composition, the fractional strength of the electromagnetic absorption by a gas is dependent on the gas concentration, the fundamental properties of the gas (the extinction coefficient, which is dependent on the wavelength) and the pathlength through the absorbing gas. The mathematical relationship between these properties is described by the Beer Lambert Law. If a low gas concentration is desired to be determined, sensitivity for a particular gas may be enhanced by choosing a strong absorption line and/or a long pathlength. However, if the absorption of light is too strong, such as if a high measurand gas concentration is present and/or long pathlength, non-linearities and/or absorption saturation may occur, reducing sensitivity with concentration change. Hence, the required sensitivity (which will determine the chosen wavelength and pathlength) is dependent on the required measurement range. The wavelength chosen for the excitation also has pragmatic considerations, such as the availability and cost of excitation sources and detectors. For example, longer wavelength infrared lasers (such as interband or quantum cascade lasers), which may have stronger absorption, may cost considerably more than near infrared lasers (such as vertical cavity surface emitting laser (VCSEL) or distributed feedback (DFB)), which operate using weaker overtone absorption bands. Also, for broadband measurements, infrared LEDs typically have increased cost and decreased output intensity, when longer wavelengths are chosen. Once the excitation wavelength or wavelength range has been selected, then the pathlength will be determined by the required measurement range. Long pathlengths may be achieved by simply using a long sample cell, but this may be (physically) impractical and with a slow time response due to the large cell volume, especially if the required pathlength is of several metres or more. Many of these issues may be mitigated, such as by using a multi-pass or folded path cell, such as a Herriott cell (FIG. 2 (a)), White cell (FIG. 2 (b)) or tuned optical cavity, which may reduce the overall cell size considerably. In FIG. 2 (a), the source of electromagnetic radiation 201 is collimated by a lens 202 and directed into the Herriot cell folded optical geometry 203. Within this cell there are two concave mirrors 204 to steer the beam in multiple folds to increase the pathlength. The detector 205 measures the transmitted radiated intensity. Gas exchange ports 206 allow the sample or calibration gas mixtures to be passed in and out of the cell. In FIG. 2 (b) the source of electromagnetic radiation 207 is collimated by a lens 208 and directed into the White cell optical geometry 209. Within this cell a set of planar mirrors 210 are used to steer the beam in multiple folds to increase the optical pathlength. The detector 211 measures the transmitted radiation intensity. Gas exchange ports 212 allow the sample or calibration gas mixtures to be passed in and out of the cell. In any of the above cases, optical treatment and/or focussing of the excitation beam will normally be required for optimal functionality, needing bespoke optical design elements, such as refractive and/or reflective elements, and precision alignment tuning is needed as part of the manufacturing process. High performance, long pathlength multi-pass or folded path cells will require the use of low temperature coefficient of expansion materials and close temperature control for best performance. In addition, multi-pass cells and optical cavities may be highly sensitive to vibration and contamination.

There are also instances where an intermediate length folded absorption pathlength may be desirable to obtain some extra absorption benefit in a more compact format, whilst not requiring the optical complexity and environmental sensitivity of a very long pathlength multi-pass cell. Novel devices addressing these issues are described in UK Patent Application No. 2304895.2. The devices disclosed in that patent application do not require beam shaping, or the combination of focusing optics with an individual optical tuning process (alignment or beam shaping), which are required by some absorption spectroscopy systems. The present invention, which is described below, may be implemented in such devices.

In some optical absorption spectroscopy systems, although the majority of the optical path between a source and a detector is within a measurement volume such as a sample cell, there is also a small part of the optical path between the radiation source and cell input optical element, and between the cell output optical element and the detector. In this patent specification, we will refer to these parts of the absorption spectroscopy system that are outside the detection/measurement volume but within the optical path as “dead volumes” or “dead zones” as the gas to be tested is not intentionally flowed into these zones. These will typically be zones that are unswept by any purge gas. If any target gas or optically interfering species is present in one of these dead volumes or zones that are within the optical path, this will have an additional absorption effect to the gas in the sample cell. This is illustrated in the earlier figures as 107 and 213. This absorption effect will be affected by environmental conditions, especially temperature. It is known to attempt to deal with “offsets” on the baseline signal, in particular through calibration, but this may still limit the lower level of detection and sensitivity. Alternatively, the dead volume may be purged with a continuous flow of non-optically absorbing purge gas, but this will have increased cost and control requirements, as well as environmental and thermal consequences.

### SUMMARY

A first aspect of the present invention provides an apparatus for use in gas absorption spectroscopy, comprising:
at least one source of electromagnetic radiation for transmitting electromagnetic radiation along an optical path that passes through a gas measurement volume, towards at least one detector;
at least one detector to detect the transmitted electromagnetic radiation after passing through the gas measurement volume and to provide an output signal indicative of the detected electromagnetic radiation; and
an analyser connected to the at least one detector to receive the output signal and analyse the effects of absorption by at least one gas species within the gas measurement volume for at least one wavelength range of the transmitted electromagnetic radiation, thereby to detect or measure a parameter of the at least one gas species;
wherein the at least one source and at least one detector are arranged in positions relative to the gas measurement volume such that there is at least one void within the optical path of the transmitted electromagnetic radiation, between the source and the gas measurement volume and/or between the detector and the gas measurement volume, wherein the at least one void is filled with an optically transmissive filler material.

The inventors of the present invention have identified a need to mitigate the problems of system costs, environmental costs and inaccuracies resulting from uncalibrated absorption by interferent gases that may leak into voids within a gas absorption spectroscopy system outside the system's measurement volume. They have mitigated these problems by filling one or more voids within the optical path of the absorption spectroscopy system with an optically transmissive filler material. The filler material can reduce or eliminate the presence of potential interferent gases outside the measurement volume, which gases could otherwise exhibit significant absorption of relevant wavelengths. Because of this, the filler material can avoid or significantly reduce unwanted absorption outside the measurement volume. This is particularly beneficial when measuring very low gas species concentrations and in compact devices for which potentially gas-containing volumes outside the gas measurement cell are a non-negligible part of the optical path length. A filler material can be selected based on desired absorption characteristics, such as low absorption for wavelengths that are known to be absorbed by a gas species that needs to be measured. We will describe selection of materials that have desired transmission of electromagnetic radiation in a wavelength range containing a characteristic absorption wavelength for the measurand gas species. Although a low absorption material is preferred for many applications, a filler material that absorbs some electromagnetic radiation can still mitigate the problem of uncalibrated variations in absorption by interferent gases such that, after calibration, the system can achieve more accurate measurement of absorption in a measurement volume.

In general, the optically transmissive filler material is not required to transmit 100% of electromagnetic radiation of all wavelengths but is selected to provide a desired transmittance of electromagnetic wavelengths of a specific wavelength range, such as in the near infrared. In one example, the filler material is selected to have low absorption for a wavelength range that is known to include one or more absorption wavelengths of a gas species which is to be detected or measured by the absorption spectroscopy apparatus. In a specific example, the selected material has high internal transmittance corresponding to low absorption and low scattering, as well as low reflection.

In an example absorption spectroscopy system, the optically transmissive filler material is selected to have low gas permeability, potentially for all gases. The filler could be selected to have low permeability to selected measurand gases (and potentially also to gases that are known to have interferent effects with selected measurand gases) for which the absorption spectroscopy apparatus is intended to be used. In this patent specification, references to a ‘gas impermeable’ or ‘low permeability’ material are intended to refer to a material that has low permeability at least for measurand gases, to mitigate the problem of interferent gases outside the measurement volume, unless stated otherwise. Although dead volumes could, in theory, be evacuated or filled with a non-optically absorbing gas, this would require a high-integrity seal, preferably with scrubbing, to avoid the risk of an interferent gas being present in the dead volume, and this adds to build complexity and cost. An optically-transmissive filler material with low gas permeability in the form of a gas impermeable optically transmissive liquid, gel or solid can mitigate the risk of interference without this build complexity and cost.

An example absorption spectroscopy apparatus includes a gas sample chamber or other gas detection and/or measurement volume for containing gases to be analysed, and includes at least one additional volume (or void) that is within the optical path of the transmitted electromagnetic radiation, wherein the additional volume is located between the at least one source and the gas detection and/or measurement volume, and/or the additional volume is located between the at least one detector and the gas detection and/or measurement volume (referred to simply as a “measurement volume” hereafter). The invention mitigates the problem of absorption within the additional volume or volumes by use of a low absorption and low permeability filler material. An example apparatus for use in absorption spectroscopy combines a source, a detector and a measurement chamber with reflective surfaces defining a folded-optical path between the source and detector. Within such an apparatus, the folded optical path may include a desired optical path length through a gas within the measurement chamber (or other gas-containing volume) and an additional part of the optical path that is between the source of electromagnetic radiation and an entry window into the measurement chamber, and an additional part of the optical path between an exit window of the chamber and a detector. Without the present invention, those parts of the optical path that are outside the gas measurement chamber are additional volumes that could contain interferent gases (either from the date of manufacture, or due to leakage after manufacture) that reduce the accuracy of absorption measurements.

The present invention can be used to reduce or eliminate the problem of absorption by gases contained within a volume (or multiple volumes) within the optical path but outside the gas detection and/or measurement volume of an absorption spectroscopy system. In an absorption spectroscopy system, there may be purged or un-purged “dead volumes” or “dead zones” of the apparatus that exist as a consequence of the particular positioning of system components. The inventors of the present invention have determined that, if these “dead volumes” are within the optical path, there is a need to mitigate the potential problem of absorption of the wavelengths of electromagnetic radiation that are of interest for detecting or measuring gases in the detection/measurement chamber of an absorption spectroscopy system. This mitigation is especially useful in a compact absorption spectroscopy apparatus in which a “dead volume” is a non-negligible part of the optical path. The potential for leakage of an interferent gas into such secondary volumes is easily overlooked. A selected low permeability and low absorption filler material can be used to reduce or eliminate the presence (in the additional volume) of at least one undesired gas species, thereby to avoid undesired absorption effects that would reduce the accuracy of gas detection and measurements. The low permeability material may be entirely transparent or semi-transparent to a desired wavelength range. In an example, at least one dead volume within an absorption spectroscopy system is filled with a gas-impermeable fluid and/or gel that is selected for its viscosity and transmittance properties, to flow into the additional volume where it will reduce or eliminate the presence of undesired gas species while transmitting the electromagnetic radiation that is required for absorption spectroscopy measurements.

In one example apparatus, the gas detection and/or measurement volume comprises a gas sample cell with at least one gas exchange port for allowing gas into and out of the gas cell, and at least one window or other optical element for allowing transmission of electromagnetic radiation of a desired wavelength range into and out of the gas cell. An example gas sample cell has a first window allowing radiation to enter the gas sample cell and a second window allowing radiation to exit the gas sample cell. A gas-impermeable low absorption filler material is located between the source and the first window, and/or between the detector and the second window. The gas-impermeable low absorption material is preferably refractive-index matched to an adjacent optical element (e.g. matched to the windows), to reduce unwanted surface reflections.

Without the present invention, there is a risk of uncalibrated absorption of electromagnetic radiation within any gas-containing secondary volume that is outside the gas detection and/or measurement volume. Specifically, referring to the example of FIG. 8, a void or dead space between the at least one source (809) and/or at least one detector (810) and the at least one optical element (806) on the sample cell may be a cause of measurement uncertainty, due to the presence of the gas species of interest or an optical interferent. This interfering gas species may be present from the time of manufacture or through diffusion from the surrounding environment and could be variable. This may be mitigated by several traditional means such as shown in FIGS. 8 (a) and 8 (b). Known solutions include flushing of the dead space with a non-optically absorbing gas, such as nitrogen (FIG. 8 (a)) and/or chemical scrubbing for a specific gas or specific gases (FIG. 8 (b)). With reference to FIG. 8, a more detailed explanation of the above options will be given (with reference to FIGS. 8 (a) and 8 (b)) together with an alternative strategy (with reference to FIG. 3 (c)). In FIG. 8 (a), the dead space is sealed by the optical elements 806 and it is purged with a non-optically interfering gas. This has the advantages of eliminating the measurement uncertainty, but has the disadvantage of needing a continuous supply of purge gas with increased complexity and cost. In FIG. 8 (b), a scrubber 807 is used to eliminate the interfering gases in the dead space. This has the advantage of not requiring a continuous supply of purge gas, but it requires a high integrity seal and the scrubber may become saturated over time requiring replacement. The scrubber material should be selected according to the application, increasing complexity and cost. Alternatively, as proposed in this patent specification and shown by way of example in FIG. 8 (c), a dead space 808 (or any secondary volume that is within the optical path but separated from the detection and/or measurement volume) may be advantageously filled with a gel, or other suitable substance such as a fluid or adhesive, which is at least partially transmissive to the wavelength range of interest and substantially impermeable to interferent gases that might otherwise absorb photons having certain energies (corresponding to particular wavelengths of the transmitted electromagnetic radiation) and thereby reduce the accuracy of measurements of absorption. The gas-impermeable transmissive material may be refractive index matched to the optical element materials of the source 809 and/or the detector 810, and/or matched to the optical elements 806 of the cell.

Additionally, the properties of the material that is used to fill dead space 808 may be required to be approximately electrically and thermally insulative. The refractive-index matching minimises stray reflections and transmission losses and decreases etalon formation. The selected material may be gas-impermeable to act as a secondary physical barrier to gas ingress/egress, in case of the failure of the sample cell optical seal. This may be particularly relevant for flammable and/or toxic gas samples. In a preferred embodiment, a gel or other suitable substance may be injected/poured or placed by a suitable means into the dead space 808 in order to completely fill the void between optical elements of the source, the detector, and the gas sample cell's optical elements 806. In a preferred embodiment, when the gel or other suitable substance is injected into the dead space, its viscosity should be chosen so as to allow complete flow access for avoidance of any bubble or void formation. This viscosity may be controlled by composition and/or temperature. Additionally, minimisation of (air) bubble formation may be enhanced by the use of a vacuum, when making and/or injecting the gel and/or setting the gel. Flexibility within the gel structure decreases the likelihood of crack formation due to aging or temperature cycling. The presence of bubbles within the gel may decrease the performance through scattering, absorption and etalon formation.

Another aspect of the invention is an optically transmissive material for use in absorption spectroscopy systems, which is selected or formulated for a desired transmittance combined with low permeability to interferent gases and high thermal stability, which material can be used for filling voids within the absorption spectroscopy system that are outside a gas sample measurement chamber but within the optical path of electromagnetic radiation that is transmitted through the gas measurement volume. The material preferably comprises a gel or a fluid, which can be flowed into a void within the system. Example materials having high transmittance for infra-red wavelengths are described below, together with a method of formulating a doped material for applications that require reduced transmittance or require refractive index matching.

Another aspect of the invention is a method of constructing an apparatus for use in absorption spectroscopy, comprising the steps of:
providing a gas cell enclosing a gas detection and/or measurement volume, the cell having at least one gas exchange port and having at least one optical element for allowing transmission of electromagnetic radiation into and out of the gas cell;
providing at least one source of electromagnetic radiation, for transmitting electromagnetic radiation along an optical path that passes through the at least one optical element into the gas detection and/or measurement volume, towards at least one detector;
providing at least one detector to detect the transmitted electromagnetic radiation after passing through the gas detection and/or measurement volume and out of the gas cell through the at least one optical element, and to provide an output signal indicative of the detected electromagnetic radiation;
arranging the gas cell between the at least one source and at least one detector such that there is a void between the at least one source and the at least one optical element, and/or a void between the at least one detector and at least one optical element; and
   flowing an optically transmissive gel or liquid or powder material into the void between the at least one optical element and the at least one source, and/or flowing an optically transmissive gel or liquid or powder material into the void between the at least one optical element and the at least one detector, thereby to reduce the space available for interferent gases in voids within the optical path.

### BRIEF DESCRIPTION OF DRAWINGS

Example systems and methods are described below with reference to the accompanying figures in which:

FIG. 1 shows a prior art arrangement of a spectroscopic absorption gas analysis system for making extractive sample gas measurements;

FIGS. 2 (a) and 2 (b) show a prior art arrangement of a spectroscopic gas analysis system with a folded pathlength by using multiple reflections, from a collimated light beam;

FIG. 3 shows an example spectroscopic gas analysis system in which the present invention may be implemented;

FIG. 4 shows an example of a spectroscopic gas analysis system with structures similar to FIG. 3, where a combination of two or more folded path cells are used to increase the overall absorption pathlength;

FIG. 5 illustrates another example gas analysis apparatus implementing an alternative optical geometry, with longer pathlength per cell length.

FIG. 6 shows the inclusion of a wavelength lock cell into a device;

FIG. 7 shows examples of a spectroscopic gas analysis system, where an electric and/or magnetic field is applied to one or more regions;

FIGS. 8 (a) and 8 (b) show examples of spectroscopic gas analysis systems, where sample gas, a purge gas or reference gas is flowed through the dead space or a scrubber is applied to the source and/or detector dead space regions;

FIG. 8 (c) shows an example spectroscopic gas analysis system with a transmissive material filling a dead space adjacent a light source and a detector;

FIGS. 9 (a), 9 (b), 9 (c) and 9 (d) illustrate a Chip-on-Board design, with a transmissive filler material shown in each of FIGS. 9 (b), 9 (c) and 9 (d);

FIG. 10 (a) illustrates the effect of residual oxygen in a void within the optical path of transmitted radiation, and FIG. 10 (b) illustrates the same apparatus with a silicone-based gel introduced into the void to displace the residual oxygen;

FIG. 11 shows a transmittance spectrum for an example filler material, which has high transmittance (almost 100% transmittance relative to air) for some wavelengths, but much lower transmittance for some other wavelengths; and

FIG. 12 shows the relationship between refractive index and wavelength for an example transmissive filler material, at 25° C.

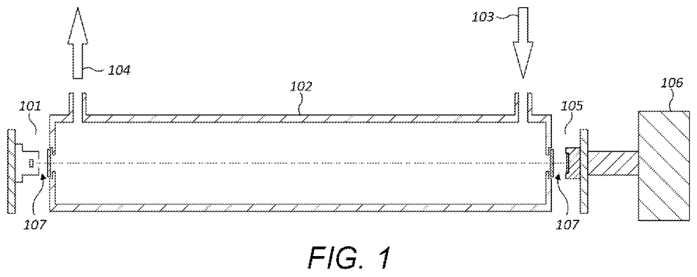

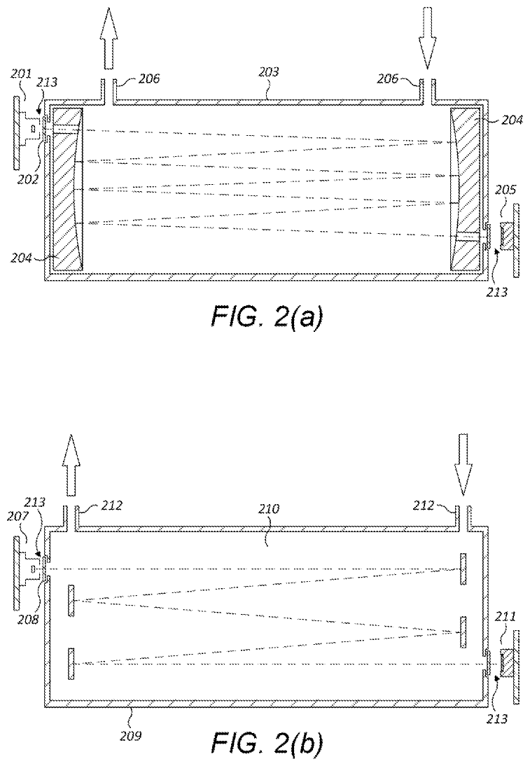

### DETAILED DESCRIPTION

This patent specification describes exemplary methods, apparatus, and systems for spectroscopic absorption measurements, as set out in the accompanying claims, whereby a gas-impermeable and transmissive filler material is added to a volume that is outside a gas detection and/or measurement volume but is within the optical path of transmitted electromagnetic radiation. For example, a gas-impermeable transparent gel may be used to fill a “dead volume” adjacent a source or detector of electromagnetic radiation. In this way, leakage or diffusion of an interferent gas into the dead volume in the absorption path is reduced or eliminated. The gas-impermeable and transmissive filler material may be in the form of a polymeric gel or a colloidal suspension of a first solid substance suspended in a liquid—each of which has a consistency enabling the dead volume to be filled without voids. In the description below, references to the example of a gel or polymeric gel should be interpreted as including other filler materials, as references to use of a gel does not preclude the use of low, medium or high viscosity fluids or adhesives, as well as a gelatinous medium.

When introduced into a void within the apparatus, the gel displaces whatever gas is present, and prevents its return. The properties of the polymeric gel are such that, once set, although there is still some liquid phase, it behaves somewhat like a solid due to the cross linkages between polymer molecules. The extent of cross linking and type of polymer will determine the rigidity and other features of the gel. The inventors have determined that the properties of the gel, as well as the means and environment under which it is placed within the dead volume, will influence its functionality within an absorption spectrometer. For example, if the gel is injected into the dead volume cavity, the occurrence of gas (e.g. air) bubbles or voids within or around the gel may be reduced if this takes place under vacuum, but also the dead volume mechanical design, orientation with respect to gravity, temperature and gelling conditions will be taken into consideration. The viscosity of the gel whilst injecting or pouring should be chosen to allow the free movement of the gel within the dead volume cavity and to fill all of the voids and make contact with all of the exposed surfaces within the optical path of the transmitted radiation. Very small spaces and/or apertures within the dead volume should be avoided whenever the apparatus design constraints allow this, as well as very high surface roughness being avoided where possible, since this may inhibit free ingress of the gel. The speed at which the gel solidifies may be influenced by composition, catalytic action and temperature.

The gel may be composed of a silicone-based material. Silicones are known to exhibit high optical transmittance for certain wavelengths, with good thermal stability (resistance to high temperatures) and flexibility. In particular, silicone formulations can be engineered to have selected transmittance for wavelength ranges of interest within the near-infrared and mid-infrared regions of the electromagnetic spectrum, making a silicone gel very suitable as the optically transmissive material for filling a void within the optical path of an infrared source within a gas absorption spectroscopy system. As an alternative to a silicone-based gel, other suitable materials include optically transmissive polymers such as cyclo-olefin copolymer (COC) which combines high thermal stability and chemical stability with good transmission of infrared wavelengths. COC optical transmittance is high and stable for wavelengths in the range 400 nm to 1100 nm. A COC-based gel can be formulated with controlled viscosity by addition of plasticizers, solvents or other additives.

Silicones and COC usually have different refractive indexes and transmission properties, and so the material selection may be based on refractive index matching with an optical element such as an entry or exit window of a gas sample chamber and/or based on their transmission properties for the wavelengths of interest. COC typically has a refractive index in the range 1.51 to 1.53, depending on the formulation, which is close to the 1.54 refractive index of quartz windows. Some silicones have refractive indexes around 1.54, but silicones can be formulated to have a wide range of refractive indices. Therefore, an absorption spectroscopy apparatus combining quartz windows and an appropriate selected or formulated silicone or COC as the filler material adjacent the quartz windows would have relatively good refractive index matching, thereby reducing surface reflections.

Other suitable materials are polymethyl methacrylate (PMMA) and polycarbonate (PC), which have high optical transmittance (low absorption) in the wavelength range 400 nm to 1100 nm and are usually formed as a rigid thermoplastic but could be combined with a solvent to create a flowable liquid which then solidifies. Another candidate material is polyethylene terephthalate (PET), which has relatively stable transmittance across a range of wavelengths (mainly within the range 600 nm to 1100 nm), although the transmittance is lower than PMMA or PC. PET could be formulated as a flowable solution by blending with a solvent or plasticizer and then evaporating the solvent, or through copolymerization followed by cooling. Alternatively, the dead volume could be filled with a polymer or other suitable material in powder form and melted in situ to fill the void. Another option is to insert a pre-moulded and/or machined glass and/or gel and/or polymer insert to replace some or all of a dead volume. This latter option could be combined with the use of a secondary filler to reduce or eliminate any remaining dead volume. Although some of the above example materials, such as PET, exhibit absorption in the infrared range, it is not essential for the present invention to have complete transparency to the relevant absorption wavelengths of the source of electromagnetic radiation, because the apparatus can be calibrated with either no gas or with a known calibration gas in the gas sample chamber. Thereafter, any absorption by the filler material will be invariant for a given wavelength. Suitable commercially available COC gels may be used such as Zeonex® or Topas® gels, and their refractive indices can be matched to quartz. Suitable commercially available silicone-based filler materials may be used, such as materials in the Avantor® NuSil® silicone gel range for near infrared applications, dependent on the required properties and spectrally transmissive range. For example, the Avantor NuSil LS1-3252 silicone gel was found to be suitable for use in an absorption spectroscopy apparatus measuring gas concentration using wavelengths in the range around 600 nm to 800 nm, which is suitable for measuring oxygen absorption lines around 760 nm, with a refractive index around 1.52 to 1.51 at 25° C., and low moisture absorption. An example of a silicone-based polymer that possesses high transmittance for infrared wavelengths and high thermal stability, and which can be formulated as a gel with a refractive index matching quartz, is polydimethylsiloxane (PDMS).

The gel may be chosen to be transparent or almost transparent to the wavelength range of interest or, conversely, the gel may be chosen so as to attenuate the radiative output from the source. Attenuation may be desirable, for example, if the optical intensity of the unattenuated radiation would saturate the detector or if the intensity could potentially cause ignition within a flammable mixture. This attenuation could be due to the intrinsic properties of the gel and wavelength used or the material could be doped with materials and/or dyes to absorb some of the radiative throughput. The gel may also act as a secondary barrier for leaks to and from the sample cell. This could be especially relevant for flammable and/or toxic samples. The gel should preferably have low permeability to relevant and/or interfering gases and be mechanically stable over time, i.e. should not shrink back or change spectroscopic properties.

Since at least one of the radiative source, detector or sample cell may require independent temperature control, typically via a TEC (thermo-electric cooler) or electrical heater, generally, the gel should have a low thermal conductivity, however, there may be instances when a close thermal coupling is desirable to create a homogeneous thermal environment, in which case a high thermal conductivity gel is needed. Since the gel may be contiguous with electronic contacts and components, the gel may preferentially be required to be non-electrically conductive.

Although the gel's primary function is to fill the dead volume and eliminate gaseous optical absorption, if the refractive index of the gel is closely matched to that of one or more of the optical elements of the absorption spectrometer (source, detector, gas cell optical inlet/outlet), it may reduce back reflection/increase transmission and hence enhance optical throughput and attenuate optical interference and optical feedback into a solid state laser (if used). Although refractive index matching gels are already used in various optical systems, such as compound lenses and fibre optic coupling to reduce interface effects between individual optical components, that implementation and purpose is very different from that being described within this patent specification. In some instances, where there is significant refractive index variation between optical components, a graded refractive index gel may be desirable to minimise reflective losses. This may be achieved by layering different gel compositions and/or through doping the material or through other suitable means.

In the special case of using a COB (Chip-on-Board) tuneable laser diode as the radiative source, such as a VCSEL (vertical cavity surface-emitting laser), DFB (distributed feedback laser) or DM (discrete mode laser), where the solid state laser is in direct contact with a transmissive filler material such as a flowable filler gel, the refractive index of the gel may be specifically chosen so as not to closely match the refractive index of the solid state laser, since this might affect the gain medium created by internal reflection at the edge of the laser structure and hence reduce or interrupt lasing action. In such a case, however, it may still be desirable to refractive index match the gel to the optical input and output optical elements of the sample cell. In the case of a COB laser, the gel may also physically protect the laser diode from electrostatic and/or mechanical damage and/or chemical attack from moisture or other chemicals present in the dead volume.

A COB implementation may also be used for a solid-state detector, such as a photodiode. Photodiodes in the near infrared, for example, may be silicon, InSb or InGaAs based or from other suitable material systems. The use of a gel in the dead volume, in conjunction with a COB solid laser diode or LED and/or photodiode detector may be especially beneficial for a compact, low-power, low-cost absorption spectrometer design.

It may be desirable to have at least one fluid (gas or liquid) reference volume encapsulated within the gel structure of the dead volume. We will refer to this as a fluid reference bubble. This may be used as a line-lock reference (potentially containing the gas of interest or other suitable gas within the wavelength measurement range), or for optical absorption interference reduction or another appropriate function. This may be achieved by injecting a known fluid into the gel using a syringe under controlled conditions (such as location, composition, temperature and pressure of the fluid) or by other suitable means. Note that the location of the gel with a reference bubble may be located close to the line-lock detector for TDL measurements, potentially eliminating the need for an alternative reference means, such as a sealed reference cuvette.

If electrical or magnetic fields are deployed across the gel, the physical and spectroscopic properties of gel in certain preferred embodiments should be only minimally affected or completely unaffected by the presence of these fields, so as to minimally affect the spectroscopic measurement.

The inventors are describing within this patent:

An apparatus for optical gas detection and/or measurement in an absorption spectroscopy system comprising:
a gas cell for containing a gas sample or calibration gas with at least one gas exchange port and at least one optical element for allowing transmission of electromagnetic radiation of a desired wavelength range in and out of the gas cell; and
at least one source of converging, diverging or collimated electromagnetic radiation for transmitting electromagnetic radiation through a gas sample contained within the gas cell and towards at least one detector;
at least one detector to monitor absorption of electromagnetic radiation for at least one absorption wavelength or wavelength range associated with at least one gas species, by detecting transmitted electromagnetic radiation that is not absorbed; and
   at least one analyser for analysing an output signal from the at least one detector to determine the presence and/or measure a parameter of at least one gas species within the gas sample,
wherein at least one void or ‘dead volume’ within the spectrometer, which void is outside the gas cell but within the optical path of the transmitted electromagnetic radiation, is filled with an optically transmissive filler material such as a fluid and/or a gel that reduces or eliminates the presence of at least one undesired gas species.

Other features and functions may also be integrated in and around the cell in order to enhance its optical performance, environmental stability and signal processing. These optional features and functions may include optical elements including windows, lenses, mirrors, attenuators, optical band pass filters, or reference cells for line locking and/or validation. Additionally, physical and spectroscopic features can be used to minimise stray optical reflections, such as using surface roughening and/or blackening, or environmental factors including gas flow, temperature and pressure may be controlled and/or corrected. In some examples, flow and/or diffusional features (such as gas ports, diffusional exchange membranes) and volume reduction features are used to reduce response time. In some examples, the application of electrical and/or magnetic fields to the measurement cell and/or other regions, and/or path modulation is used to reduce etalon effects. Signal processing, including frequency domain, filtering and averaging techniques may also advantageously be employed.

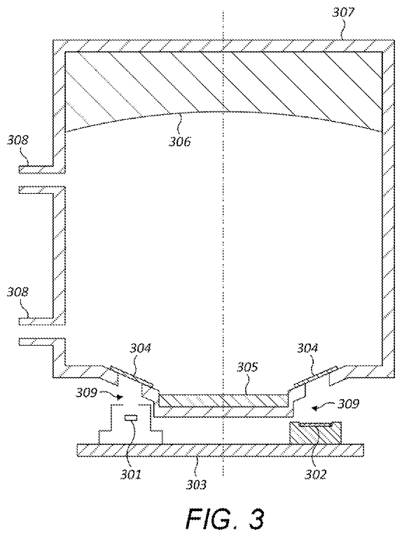

As mentioned above, the invention may be implemented, for example, in an absorption spectroscopy apparatus including a folded-path gas sample cell, as described in UK Patent Application No. 2304895.2. As illustrated in the example of FIG. 3 of 2304895.2, a compact absorption spectroscopy apparatus may include at least one source (301) of electromagnetic radiation for transmitting electromagnetic radiation through a gas sample contained within the gas cell (307) and towards at least one detector (302). The gas cell (307) for containing a gas sample or calibration gas has at least one gas exchange port (308), at least one optical element (304) for allowing transmission of electromagnetic radiation of a desired wavelength range in and out of the gas cell, and two or more mirrors (305, 306) arranged in opposed relation to each other, including at least one curved mirror (306) arranged to reflect the transmitted electromagnetic radiation towards a second mirror (305), wherein the second mirror (305) is arranged to reflect the electromagnetic radiation back towards the at least one curved mirror, and wherein the two or more mirrors (305, 306) are arranged to reflect the electromagnetic radiation in a folded optical path through the gas sample towards the at least one detector (302) such that a transmitted beam of electromagnetic radiation is directed towards the at least one detector (302). This arrangement using one or more curved mirrors can provide automatic focussing of a transmitted diverging beam such that it converges towards the detector after being reflected within the gas cell. However, the apparatus can also be used with a laser source whose collimated beam is reflected towards the detector; the curved mirrors provide a degree of tolerance to manufacturing variations both for diverging and collimated beams. The at least one detector (302) monitors absorption of electromagnetic radiation for at least one absorption wavelength or wavelength range associated with at least one gas species, by detecting transmitted electromagnetic radiation that is not absorbed and at least one analyser is used for analysing an output signal from the at least one detector to determine the presence and/or measure a parameter of at least one gas species within the gas sample.

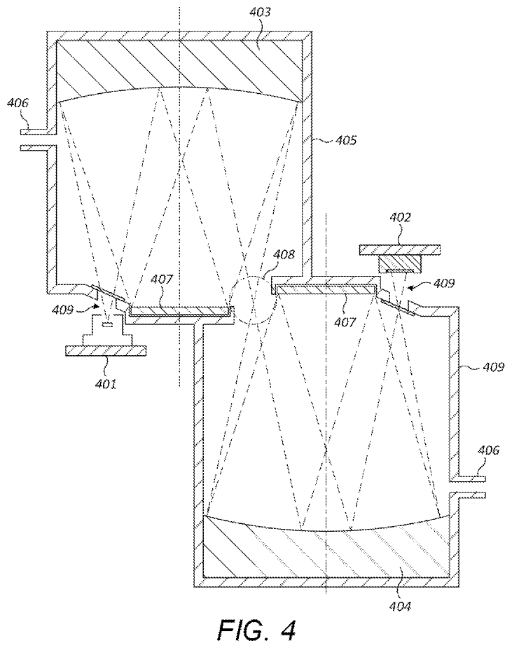

FIG. 4 illustrates a modular implementation of the device from FIG. 3. In the embodiment of FIG. 4, radiation from the source (401) enters the sample cell (405) and is reflected by a first curved or spherical mirror (403) onto a second mirror (407), back onto the first mirror (403), which then focuses the light as it exits the first cell (408) which then becomes the input into the second cell (409). In a likewise manner, the light is reflected by the mirrors (404) and (407) within the cell (409) and is focused onto the detector (402). Sample gas is allowed to pass through the cell via inlet and outlet ports (406).

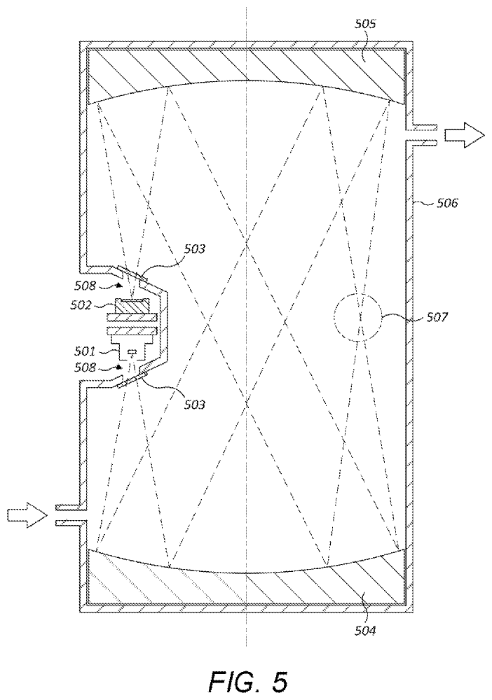

Another preferred embodiment from UK Patent Application No. 2304895.2 is illustrated in FIG. 5, for an embodiment with a longer pathlength per cell length. This embodiment achieves an enhanced pathlength with simplicity of design and build compared to a modular system of two of the earlier-described embodiments (FIG. 4). The diverging beam from the source (501) passes through the optical element (503) and enters the sample cell (506). Inside the sample cell multiple reflections occur between the first mirror (504) and a second mirror (505) as illustrated in FIG. 5. In such a simplified embodiment, mirrors (504) and (505) may be identical cross-sectional slices of a spherical section. The outgoing beam passes through the optical element (503) and is incident on the detector (502). A wavelength lock cell may be included, which may be preferentially located at position (507), where a localised focal point forms and an optical element may be used to divert a small fraction of the beam through an optionally placed wavelength lock cell.

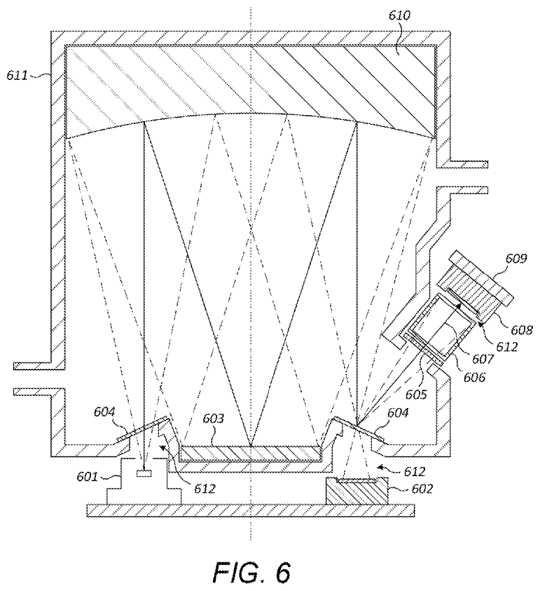

The stability of the scan current and the laser diode characteristics are paramount to the wavelength stability of the TDLS measurement. It is vital that in the case of either electronics or laser diode drift over time, the measurement can not only remain “locked” to the desired absorption line but also produce a diagnostic report about the extent of this drift as a measure of preventative maintenance. A simple approach would be to enable an algorithm that detects the location of the gas absorption 2nd harmonic peak, and actively maintains this location in a feedback loop, by continuously fine adjusting the set point temperature of the laser diode. However, this approach assumes that the process gas flowing through the cell always contains some desired gas, which produces a measurable 2nd harmonic peak. In practice, no such assumption can be made. Real life processes may be highly variable and, for long periods of time, may contain either no desired gas, or, even worse, may contain a gas with a neighbouring interfering absorption line. For this reason, the instrument is ideally fitted with an on-board “wavelength reference” device, which, in a preferred embodiment, constantly monitors a real spectroscopic 2nd harmonic signal from a known reference gas, which is always present regardless of the process variations. Ideally, this reference gas can be the main gas of interest or another surrogate gas with absorption lines nearby. For practical cost and size constraints, this reference device should be as compact and low cost as possible. FIG. 6 illustrates one of the preferred embodiments of a TDLS instrument, where the diverging beam from the source (601) passes through the optical element (604) and is then reflected by the first (610) and second mirrors (603) and, after passing the second optical element (604), is focused on the detector (602). This detector (602) is used for the primary measurand detection. In a preferred embodiment, a small proportion of the light incident on the optical element (604) in front of the detector (602) is reflected through a low volume sealed gas capsule (cuvette) (606), with transmissive optical elements (605) to the relevant wavelength range and filled with up to 100% concentration of the reference gas (607) onto a secondary detector (608) mounted on a printed circuit board (609) for line lock to be maintained. It is important that sufficient optical absorbance of the reference gas in the reference cell takes place to create a detectable 2nd harmonic signal. This is achieved by a combination of sufficient pathlength and gas fill pressure.

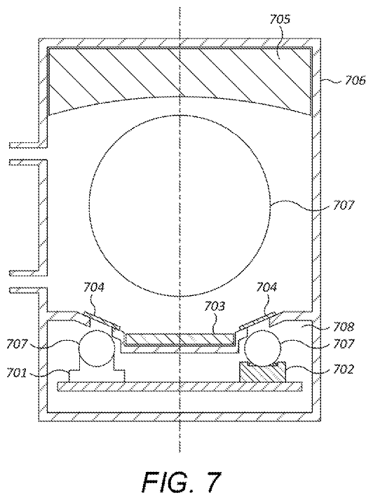

As previously described, magnetic and/or electric fields (FIG. 7) may be applied to any external volume or zone of the apparatus that is outside the detection/measurement volume but within the optical path, and/or may be applied to the gas detection/measurement volume. The diverging beam from the source (701) passes through the optical element (704) into the sample cell (706), where it is reflected by the first (705) mirror and the second mirror (703) and focused through a second optical element (704) onto the detector (702). Magnetic and/or electrical fields may be applied (707) over the external volumes (or “dead zones”) and/or over the sample cell regions. The presence of a magnetic and/or electric field may induce splitting of the absorption lines by the Zeeman and/or Stark effects, which may be used advantageously for enhanced signal processing.

As noted above, absorption spectroscopy is known for use in gas analysis, including for determining the presence of at least one particular gas species in a measurement volume and for measurement of parameters including concentration of the individual gas species in a gas sample. The use of reflective optics has advantages over refractive optics for several reasons including having a higher optical throughput (greater transmission) and being independent of wavelength (chromatic aberration), which is especially important for a modular system for the detection of many different gas species at different wavelengths. However, refractive optics may still be usefully employed in many embodiments.

Multi-gas measurements may also take place, where two or more lasers and/or detectors are used and the wavelengths of the sources are chosen to correspond to the absorption wavelengths of interest for at least two different measurands. Likewise, the detectors are chosen to have responsivities in the wavelengths of interest for the measurands. In some applications, more than two matched pairs of sources and detectors may be used depending on the measurands and the sizes of the components and the optics used.

In some preferred embodiments, signal processing may take place using analogue and/or digital electronics, including the use of multiplexed ADCs and/or processors.

Any reflective surfaces used may be made of machined and/or moulded metal, glass or polymer and polished as required and may have reflective and/or protective coatings such as gold. Any refractive optical elements used may be made of machined or moulded polymer or glass and polished as required and may have optically protective and/or anti-reflective and/or absorptive coatings. It may become important, especially for coherent sources, such as lasers, to suppress stray reflections from being collected at the detector, since constructive and destructive interference effects (etalons) could occur. The suppression of these stray reflections may take the form of random surface roughening to disrupt specular reflection and/or blackening (optically absorptive coating) of the relevant surfaces. Any such applied optically absorptive coating or material should ideally be chemically compatible with the gas sample that it is in contact with and approximately a perfect light absorber at the optical wavelength range being used. Alternatively, in some preferred embodiments, for example, with broadband (e.g. for non-coherent LED or incandescent) sources, stray reflections which arrive at the detector may actually be useful, to increase the overall optical throughput and give improved signal to noise and therefore no suppression of reflections is needed and/or these stray reflections may even be enhanced, for example, by the application of a reflective layer to at least one surface within the sample cell, such as a gold layer.

Any interaction with at least one optical element or reflective feature such as an attenuator, pass band filter, window, lens, polariser (e.g. for use if not all the laser output is intrinsically polarised) or reflective surface may cause an etalon to form. In general, the number of optical elements should be kept to a minimum, to minimise etalon formation, but there will always be some elements or features present, such as windows for the source and/or detector and for light entering into and exiting the sample cell. In some applications, where the stray light reflections should be minimised as discussed in the previous section, the formation of such etalons may be minimised by the use of anti-reflective coatings and/or angled and/or wedge windows. In addition, dimensional changes may cause shifts in the etalons and hence affect the signal. This can be reduced by the use of low thermal expansion coefficient materials such as invar and/or using a temperature-controlled cell. The use of a temperature-controlled cell and/or pre-equilibrator for the sample gas, so the incoming gas is in equilibrium with the cell temperature will increase signal stability.

The magnitude of the etalons and changes with temperature may be reduced by the use of at least one pathlength modulator. The at least one modulator may be employed at different locations within the system, depending, amongst other considerations, on the locations of the most relevant etalon producing features and the overall opto-mechanical arrangement. The modulator may be a solid-state device, such as a piezo-electric device, whose dimensions may be changed via the application of a voltage, although consideration of the voltage magnitude should be taken into account for potentially flammable mixtures. Alternatively, the modulator may be an electromechanical device, such as a voice coil type arrangement in combination with a permanent magnet, similar in design to a loudspeaker or an electromechanical vibrating element. A mechanical flexure may be employed, such as a spring or elastic polymer or foam, to act as a director and damping element. In the case of a foam being used, a sealed cell format may be preferentially employed to modify the physical properties of the flexure such as density and/or clastic properties without entraining sample gas. In some embodiments, modulations on pathlength may be preferentially applied to the approximately spherical mirror slice cross-section (306), since this has less influence on the focussing position at the detector than if the approximately flat or concave mirror (305) were modulated. The frequency of any such modulation should take into account the functional scan speed in the case of a diode laser measurement or pulse rate in the case of LED's or pulsed incandescent sources and the expected response time, so as not to detrimentally influence the measurement. The amplitude of the modulation should ideally be greater than the wavelength of light being used, however the frequency and amplitude may be chosen theoretically and/or empirically to give the desired performance and/or lifetime of the modulating element.

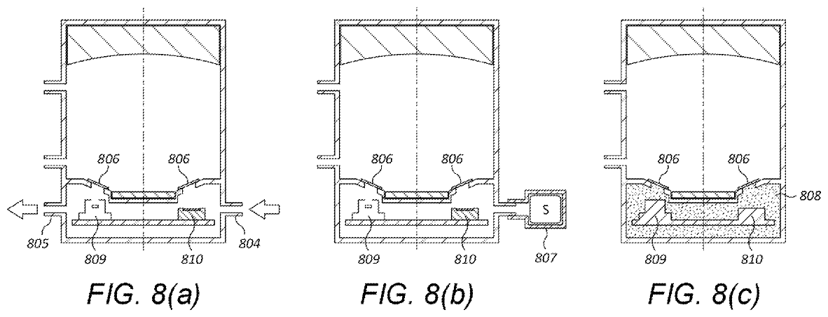

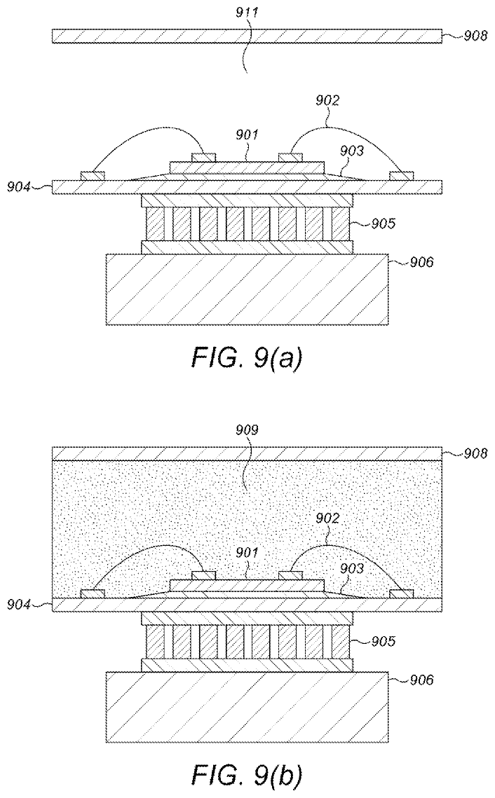

The use of a bare laser device in its native diverging cone of light chip format may be advantageous in some embodiments. There are several advantages to using the Chip-on-Board (COB) format for the laser device such as reduced cost, less packaging material, lower ctalons, higher optical transmission and significantly reduced heating and/or cooling power needed to maintain the laser chip at an accurate setpoint temperature and tighter temperature control, due to the lower thermal mass and higher thermal transfer efficiency. Chip-on-Board (COB) technology describes the mounting of the bare VCSEL or laser chip in direct contact with the substrate or (copper) plane of the PCB (FIG. 9). Among the advantages of COB are compactness, provision of the best thermal coupling of the laser 901 to the thermo-electric cooler (TEC) 905, and the elimination of any optical fringes formed inside a conventional metal can-window enclosure of a laser diode. The COB process consists of three main manufacturing processes. The first is the die mounting step or “die attach”, which consists of applying a special conductive adhesive 903 to secure the chip directly to the PCB substrate 904. The second is the “wire bonding” process which makes electrical connections 902 between the laser chip and PCB pads. In some embodiments, a third process is “encapsulation”, which consists of dispensing a very thin layer of a clear protective material over the die and the wire bonds. In some embodiments, an optically transmissive filler material 909 can be used to completely fill the void 911 between the light source and one or more optical elements such as entry and exit windows 908 of the gas cell. This filler may negate the need for the thin protective layer, or may overlay a protective layer. This optically transmissive filler material may be approximately refractive index matched to the optical elements 908, which may be one or more of the following: a window, an attenuator, a band-pass filter. Heat generated by the TEC 905 is dissipated by a suitable heatsink 906. Due to the small size of the laser chip and its direct thermal contact with the PCB copper areas, a significant reduction is achieved in the amount of TEC power required to maintain the laser chip at a desired setpoint temperature. Additionally, the significantly lower thermal capacity of the bare chip allows for very fast and accurate thermal tuning of the laser temperature and its wavelength. Additionally, the properties of the encapsulant or dead volume filler 909, where provided, should be approximately electrically and thermally insulative, since it is contiguous with the COB component and/or other electronic components. The above-mentioned approximate refractive index matching of the encapsulant or dead volume filler 909 to at least one optical component 908 reduces stray reflections and transmission losses, and decreases etalon formation and optical feedback into the laser. The encapsulating filler material may be chosen to specifically avoid a refractive index match to the laser source, because this may interfere with the laser gain medium of the COB component. The transmissive encapsulating filler material 909 may be a gel, or fluid, or may include a solid insert, and may be formed of any of the filler materials described earlier in this patent specification or another suitable filler substance, and may act as a secondary physical barrier to gas ingress/egress, in case of the failure of a seal between the COB component and the sample cell. This may be particularly relevant for flammable and/or toxic gas samples. For reasons described earlier and shown in the example of FIGS. 9 (c) and 9 (d), there may be times when it is preferable to mount the source (laser or LED) chip and/or detector chip on a sub-mount 910. This may be for at least one of the following reasons: case of handling, pre-testing of the chip, orientation, angle or thermal properties. This requires some extra wire bonding to mount the chip to the sub-mount as well as from the sub-mount to the PCB. As shown in FIG. 9 (d), the mount may have a tapering shape (with non-parallel top and bottom surfaces) for a transmission direction that is not perpendicular to the PCB substrate 904. Adhesive layers 903 have been omitted from FIGS. 9 (c) and (d), but adhesive layers may be used between the components shown in those figures. As shown in FIGS. 8 (c) and 9 (d), the windows that allow electromagnetic radiation to enter or leave the gas cell may be tilted relative to the mounted source or detector chip, for example at or close to the Brewster angle to minimise surface reflections.

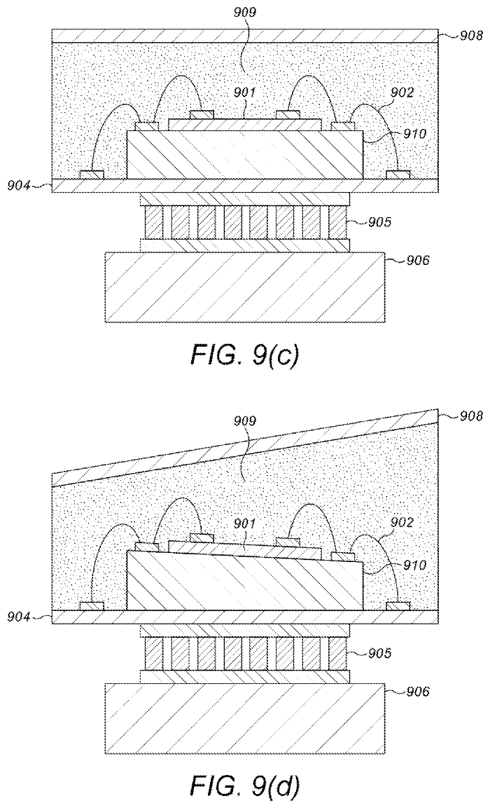

FIG. 10 illustrates the beneficial effect of injecting an example commercially available silicone-based gel (Avantor NuSil LS1-3252) into the physical apparatus shown in FIG. 8 (c), with a non-absorbing gas (nitrogen) flowing through the sample cell. In this illustration, the target gas (oxygen) is detected by scanning the electromagnetic radiative output from a Chip on Board VCSEL across an oxygen absorption line, by tuning the laser drive current and analysing the signal using WMS (wavelength modulation spectroscopy). FIG. 10 (a) shows the laser intensity reference signal 1001, and the absorption signal due to residual oxygen present in the dead volume (when no gas-displacing filler material is present) is also shown by the arrow 1002. FIG. 10 (b) shows the laser intensity reference signal 1003 and the absorption signal due to residual oxygen present in the dead volume 1004 after the optically transmissive filler gel has been injected into the cavity. It can clearly be seen that the gel has allowed transmission of electromagnetic radiation through the dead volume at this wavelength range with less absorption than in FIG. 10 (a). This is because the gel has displaced oxygen from the dead volume. For case of comparison, both FIGS. 10 (a) and 10 (b) show zoomed-in features of the oxygen absorption zone.

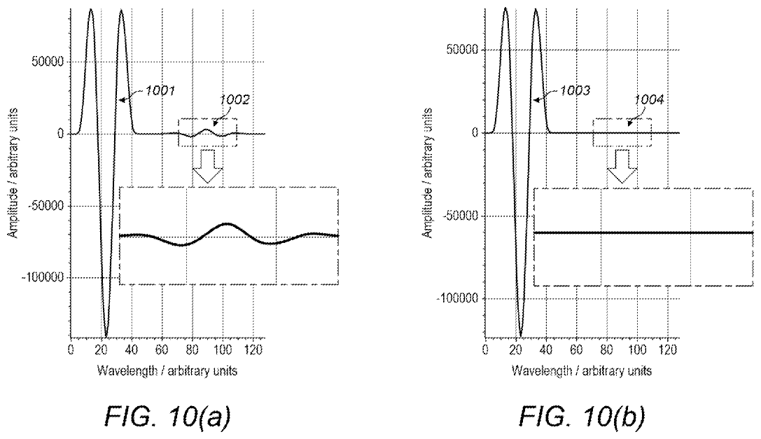

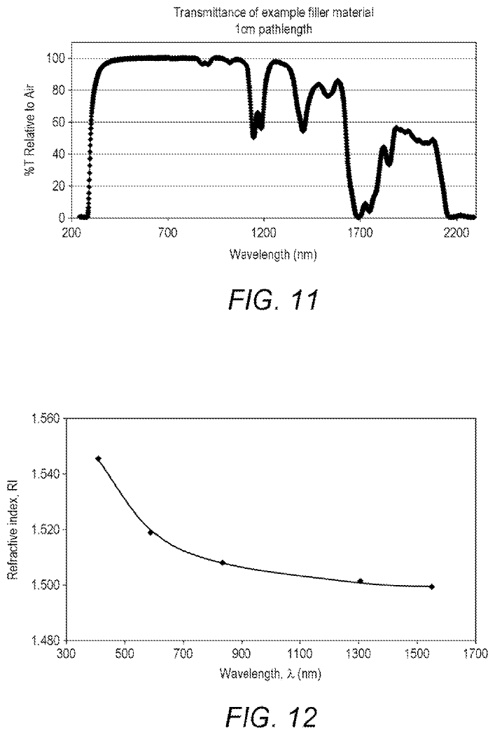

FIG. 11 is a qualitative approximation to a transmittance spectrum obtained for some samples of a commercially available optically-transmissive gel. It should be noted that the gel has high optical transmittance (approximating 100%) for some wavelengths such as around 700 nm, but the transmittance is greatly reduced for certain other wavelengths, such as around 1200 nm, 1700 nm and 2200 nm. The example gel was found to be suitable for gas measurements using electromagnetic wavelengths around 600 nm to 800 nm, but would not be suitable for measurements around 1700 nm where transmittance is low and where the variability of transmittance with small changes in wavelength is high. The graph in FIG. 11 is not intended to provide precise transmittance spectrum data, but only to illustrate the importance of selecting a filler material that is suitable for the electromagnetic radiation wavelengths to be used. FIG. 12 shows the relationship between refractive index and wavelength at 25° C., for the same material as FIG. 11.

As illustrated in FIG. 11, selecting an optimal optically transmissive filler material is an important consideration when constructing an absorption spectroscopy apparatus of the type described. Matching the filler's properties to the wavelength range of interest is important for reducing unwanted signal losses and spectral interference. Additionally, factors such as refractive index compatibility, thermal properties, and robustness to manufacturing variations all significantly impact performance. Conventional experimental and theoretical methods for material selection and characterisation can be time-consuming and may not easily account for interdependencies between material properties and ultimate system behaviour. Artificial Intelligence and Machine Learning (AI/ML) can help to address these shortcomings. Specifically, AI/ML models trained on data combining material properties, spectral characteristics, and measured performance metrics of prototype apparatuses built with varying gel formulations can be used to facilitate holistic evaluation of suitable filler materials. Principally, models trained on such datasets can be used to identify application-specific optimised filler materials, or to identify material formulations or blends without necessarily performing extensive experimentation. The same dataset may be used to generate AI/ML models which can then be applied to monitor production processes that enable early detection of variations in the gel, prompting real-time corrective actions to ensure consistent product quality and apparatus performance. The speed of response for the ML model applications mentioned above has been found to be acceptable, and comparable with some other sensors. Model explainability is not an absolute requirement for this type of application, despite being desirable. Therefore, relatively complex and abstruse models and algorithms may be employed in some applications, such as using deep, convolutional or recurrent neural networks and gradient boosted trees. These models may take advantage of distributed training/inference architecture such as Graphical Processing Units (GPU's), Tensor Processing Units (TPU's) and/or data-processing clusters, which may be hosted on a “cloud” or another distributed network.

Despite careful material selection, manufacturing or environmental factors can introduce slight variations in the properties of the optically transmissive filler material. These variations, such as subtle alterations in refractive index or absorption, have the potential to reduce the accuracy of gas absorption measurements. Conventional absorption spectroscopy apparatus lacks mechanisms for real-time monitoring and compensation of such variations, which can lead to erroneous gas concentration readings. Variations in the filler material can produce discernible and quantifiable changes in the transmitted light spectrum which can be codified in the dataset and models described above. Therefore, the present invention may employ monitoring, for example using artificial intelligence (AI), for in-situ characterisation of the optically transmissive filler material within a functioning apparatus. By continuously analysing the transmitted light spectrum through the apparatus, an AI/ML model can identify and quantify variations in the filler properties, and enable compensation for known variability of transmittance across a wavelength range. Thus, use of an absorption spectroscopy apparatus may involve monitoring the transmission of electromagnetic radiation through the optically transmissive filler material; modelling variations in transmittance of the optically transmissive filler material across a wavelength range; and performing absorption spectroscopy measurements within the wavelength range using the absorption spectroscopy apparatus, including compensating for the modelled variations in transmittance across the wavelength range. This ensures measurement accuracy is maintained, accounting for subtle drifts in filler properties over time or across different manufactured units. Real-time execution of AI algorithms within a potentially resource-constrained, microcontroller-based apparatus presents a technological challenge, but the computational demands of the chosen model can be balanced against the desired accuracy to provide accuracy improvements without excessive processing requirements. Hybrid architectures could be implemented where the microcontroller handles real-time data acquisition and preprocessing, while more computationally intensive spectral analysis and AI-based correction are performed on a remote server or in the cloud. Such approaches, however, are generally less favoured than local processing for the many users of gas sensors that do not wish to stream commercially sensitive data to external data processors. Additionally, where a sensor is to be used in a safety critical application, model explainability is paramount, which can mitigate against use of the more complex and accurate model architectures described above. Correspondingly, it is preferred that edge models employed in real time are relatively simple, computationally undemanding, and explainable, and may take the form of linear models, support vector machines or decision trees. Where neural networks are used, techniques such as quantisation and pruning may be used to reduce their computational and memory footprint, and specialised hardware accelerators may be used, such as coral.ai's TPU edge co-processor or Nvidia's Maxwell Edge GPU.

Although the above section has described the advantages and implementation of COB for a laser diode source, other embodiments may use a COB diode laser, LED source and/or solid-state detector.

When designing the opto-mechanics of the sample cell and related optics there are two important considerations to take into account:
It should provide optimum path length for the specific gas range, whilst, ideally, having a small sample flush volume for a fast response time.
It should produce a low level of optical fringes, when used with a diverging coherent source such as a laser diode.

In a preferred embodiment of a TDLS measurement system, the laser diode temperature is controlled by a thermo-electric cooler (TEC) and temperature feedback is provided by a temperature sensor, such as a thermistor, resistance temperature detector (RTD) or thermocouple. Both the laser temperature and current are controlled electronically to enable scanning over the desired gas absorption line. The detector output, which incorporates the photo detector and amplifiers, is used with real-time signal processing software, which makes use of the 2nd harmonic signal and its unique absorption shape characteristics, such as height and width to determine the true gas concentration.

It is envisaged by the inventors that there are potentially many different embodiments using the design principles described in this patent to form a gel-filled dead space with an absorption spectrometer.

In some embodiments, the source and/or detector is remote-mounted and the electromagnetic radiation is conducted to and from the apparatus using at least one fibre optic cable. This may have advantages where temperature and/or electromagnetic interference might render direct coupling impractical.

What is claimed is:

1. An apparatus for use in absorption spectroscopy, comprising:
at least one source of electromagnetic radiation for transmitting electromagnetic radiation along an optical path that passes through a gas measurement volume, towards at least one detector;
at least one detector to detect the transmitted electromagnetic radiation after passing through the gas measurement volume and to provide an output signal indicative of the detected electromagnetic radiation; and
an analyser connected to the at least one detector to receive the output signal and analyse the effects of absorption by at least one gas species within the gas measurement volume for at least one wavelength range of the transmitted electromagnetic radiation, thereby to detect or measure a parameter of the at least one gas species;
wherein the at least one source and at least one detector are arranged in positions relative to the gas measurement volume such that there is at least one void in the optical path, between the source and the gas measurement volume and/or between the detector and the gas measurement volume, wherein the at least one void is filled with an optically transmissive filler material.

2. An apparatus according to claim 1 for detecting or measuring a measurand gas species, wherein the optically transmissive filler material comprises a material that is transmissive for electromagnetic radiation in a wavelength range containing a characteristic absorption wavelength for the measurand gas species.

3. An apparatus according to claim 1, wherein the optically transmissive filler material comprises a gas impermeable liquid, gel or solid material.

4. An apparatus according to claim 1, wherein the optically transmissive filler material comprises a silicone-based material or an optically transmissive polymer.

5. An apparatus according to claim 4, wherein the optically transmissive polymer comprises cyclo-olefin copolymer (COC), polymethyl methacrylate (PMMA), polycarbonate (PC), or polyethylene terephthalate (PET).

6. An apparatus according to claim 1, wherein the gas measurement volume is provided by a gas cell for containing a gas sample or calibration gas, the gas cell having at least one gas exchange port and at least one optical element for allowing transmission of electromagnetic radiation of a desired wavelength range in and out of the gas cell.

7. An apparatus according to claim 1, wherein the optically transmissive filler material is electrically insulative.

8. An apparatus according to claim 1, wherein the optically transmissive filler material is thermally insulative.

9. An apparatus according to claim 1, wherein the optically transmissive filler material is thermally conductive.

10. An apparatus according to claim 6, where the at least one void in the optical path comprises a volume within the apparatus between said at least one optical element and the source and/or the at least one void comprises a volume within the apparatus between said at least one optical element and the detector.

11. An apparatus according to claim 10, wherein the optically transmissive filler material and the at least one optical element are selected to have approximately equal refractive indexes.

12. An apparatus according to claim 1, wherein at least one fluid reference volume is encapsulated within the optically transmissive filler material.

13. An apparatus according to claim 12, wherein the fluid reference volume comprises a line-lock reference.

14. An apparatus according to claim 12, wherein the reference volume is provided by injecting a known fluid into the optically transmissive filler material.

15. An apparatus according to claim 1, further comprising a field generator for applying electrical and/or magnetic fields across the optically transmissive filler.

16. An apparatus according to claim 1, wherein at least one source and/or detector are mounted on a circuit board substrate in a chip-on-board format and encapsulated by the optically transmissive filler.

17. An apparatus according to claim 1, wherein the optically transmissive filler material is doped with a dye or absorptive substance to attenuate the intensity of the transmitted light.

18. An apparatus according to claim 1, wherein the source of electromagnetic radiation is a broadband source.

19. An apparatus according to claim 18, wherein the broadband source is an LED or incandescent source.

20. An apparatus according to claim 1, wherein the source of electromagnetic radiation is a tuneable laser diode.

21. An apparatus according to claim 1, wherein the detector is a bolometer, pyrometer, photomultiplier or photodiode.

22. An apparatus according to claim 1, wherein the at least one source and/or at least one detector are mounted on a circuit board in a Chip-on-Board format.

23. A method of constructing an apparatus for use in absorption spectroscopy, comprising the steps of:
providing a gas cell enclosing a gas detection and/or measurement volume, the cell having at least one gas exchange port and having at least one optical element for allowing transmission of electromagnetic radiation into and out of the gas cell;
providing at least one source of electromagnetic radiation, for transmitting electromagnetic radiation along an optical path that passes through the at least one optical element into the gas detection and/or measurement volume, towards at least one detector;
providing at least one detector to detect the transmitted electromagnetic radiation after passing through the gas detection and/or measurement volume and out of the gas cell through the at least one optical element, and to provide an output signal indicative of the detected electromagnetic radiation;
arranging the gas cell between the at least one source and at least one detector such that there is a void between the at least one source and the at least one optical element, and/or a void between the at least one detector and at least one optical element; and
flowing an optically transmissive filler material, comprising a gel or liquid or powder material, into the void between the at least one optical element and the at least one source, and/or flowing an optically transmissive filler material, comprising a gel or liquid or powder material, into the void between the at least one optical element and the at least one detector.

24. A method according to claim 23, wherein an optically transmissive gel or liquid is flowed into said void or voids in the presence of a partial vacuum, thereby to minimise bubble formation within the optically transmissive filler material.

25. A method according to claim 23, wherein an optically transmissive filler material is flowed into said void or voids in powder form and the powder is melted and fused in situ to fill the void or voids.

26. A method according to claim 23, wherein a pre-moulded and/or machined optically transmissive filler insert is inserted into the void, prior to flowing an optically transmissive gel or liquid or powder material into the void.

27. A method according to claim 23, further comprising:
monitoring the transmission of electromagnetic radiation through the optically transmissive filler material;
modelling variations in transmittance of the optically transmissive filler material across a wavelength range; and
performing absorption spectroscopy measurements within the wavelength range using the absorption spectroscopy apparatus, including compensating for the modelled variations in transmittance across the wavelength range.

28. A method according to claim 23, wherein at least one fluid reference volume is encapsulated within the optically transmissive filler material.

29. A method according to claim 28, wherein the fluid reference volume comprises a line-lock reference.

30. A method according to claim 28, wherein the fluid reference volume is provided by injecting a known fluid into the optically transmissive filler material.

31. A method according to claim 23, wherein the optically transmissive filler material and the at least one optical element are selected to have approximately equal refractive indexes.

32. A method according to claim 23, wherein the optically transmissive filler material is formulated to attenuate the intensity of transmitted electromagnetic radiation entering the gas cell.

33. A method according to claim 32, wherein the optically transmissive filler material is doped with materials and/or dyes that absorb electromagnetic radiation.

34. A method according to claim 23, wherein gas absorption is measured using direct absorption spectroscopy.

35. A method according to claim 23, wherein gas absorption is measured using wavelength modulation spectroscopy.
